(19) United States
(12) Patent Application Publication
(10) Pub. No.: US 2016/0262699 A1
(43) Pub. Date: Sep. 15, 2016

(54) Endotracheal Tube for Nerve Monitoring

(71) Applicants: Andrew C. Goldstone, Baltimore, MD (US); Raymond L. Schettino, Atlanta, GA (US)

(72) Inventors: Andrew C. Goldstone, Baltimore, MD (US); Raymond L. Schettino, Atlanta, GA (US)

(21) Appl. No.: 14/836,057

(22) Filed: Aug. 26, 2015

Related U.S. Application Data
(60) Provisional application No. 62/131,931, filed on Mar. 12, 2015.

(51) Int. Cl. A61B 5/00; A61B 5/0492; A61M 16/04; A61B 5/04

(52) U.S. Cl. CPC A61B 5/6852; A61B 5/4041; A61B 5/04001; A61B 5/0492; A61B 5/7405; A61B 5/742; A61M 16/04; A61B 2562/0209

(57) ABSTRACT

An endotracheal tube for nerve monitoring including one or more ground, reference, or laryngeal-monitoring electrode wires that run in a direction parallel to the central axis of the tube, each such electrode wire having an insulated first wire portion and an uninsulated second wire portion, and further including one or more ground, reference, or hypoglossal-monitoring electrode wires wrapped around the outer surface of the endotracheal tube in a helical, circular, or interposed pattern

### CROSS-REFERENCE TO RELATED APPLICATION

The present application claims the benefit of priority of U.S. Provisional Application No. 62/131,931 filed Mar. 12, 2015. The entire text of the priority provisional application is incorporated herein by reference in its entirety.

### FIELD OF THE DISCLOSURE

This disclosure relates generally to an endotracheal tube for nerve monitoring, and more specifically, to an endotracheal tube having one or more of ground wire(s), reference wire(s), or laryngeal-monitoring electrode wire(s) running in a direction parallel to the central axis of the tube and one or more of hypoglossal-monitoring wire(s), ground wire(s), or reference electrode wire(s) distributed in a helical, circular, or interposing pattern around the tube.

### BACKGROUND

Neck surgery poses a risk to both the laryngeal nerves (which control the laryngeal muscles) and the hypoglossal nerves (which control the intrinsic muscles of the tongue). If a laryngeal nerve is damaged during surgery, paralysis of the laryngeal muscles can occur, causing a loss of speech, disrupted breathing, and/or swallowing difficulties. If a hypoglossal nerve is damaged, a loss of innervation to the musculature of the tongue may result in an inability of the tongue to move or change its shape and bulk.

The state of the art in intra-operative laryngeal nerve monitoring is based upon the invention disclosed U.S. Pat. No. 5,024,228, the entirety of which is hereby incorporated by reference. According to U.S. Pat. No. 5,024,228, an electrode endotracheal tube may be provided having electrode wires running in a direction parallel to the central axis of the endotracheal tube and exposed portions of the wires located adjacent to the laryngeal muscles when the endotracheal tube is inserted into the trachea. At present, intra-operative monitoring of the hypoglossal nerve is not generally performed during operations that put that nerve at risk. In general, any standard electro-physiologic nerve monitoring requires the use of additional patient ground and/or reference electrodes.

### SUMMARY

An endotracheal tube for nerve monitoring is hereby disclosed. The endotracheal tube includes a flexible tube having a distal and a proximal end. The tube contains a main lumen for ventilating the lungs, an inflatable cuff surrounding the tube to prevent air from escaping by passing between the tube and trachea wall, and a thin lumen for inflating the cuff.

The tube contains one or more ground, reference, or laryngeal-monitoring electrode wires that run in a direction parallel to the central axis of the tube. Each ground, reference, or laryngeal-monitoring electrode wire is insulated against electrical contact at a first wire portion located between the ends of the tube. Insulation may be achieved by coating the first wire portion or embedding the first wire portion within the wall of the endotracheal tube. An uninsulated second wire portion, located between the tube's distal end and the first wire portion, lies exposed on the surface of the endotracheal tube, permitting electrical contact to be made by the second wire portion.

In some embodiments, the tube may further contain one or more ground, reference, or hypoglossal-monitoring electrode wires wrapped around the outer surface of the endotracheal tube in a helical pattern. Electrode wires dedicated to the same purpose (ground, reference, or hypoglossal-monitoring) may be used to form the helical pattern or a combination of the various purposed electrode wires may be used. The helical pattern of each ground, reference, or hypoglossal-monitoring electrode wire is identical, and each ground, reference, or hypoglossal-monitoring electrode wire is offset from every other electrode wire having a helical pattern by a longitudinal distance along the tube, such that a constant distance is maintained between each electrode wire having a helical pattern. In short, no crossing of electrode wires with helical patterns occurs. The helical pattern of each electrode wire begins closer to the proximal end of the uninsulated second wire portion than to the proximal end of the endotracheal tube, wraps around the endotracheal tube over the insulated first wire portion, and terminates closer to the proximal end of the endotracheal tube than to the proximal end of the uninsulated second wire portion.

In other embodiments, the tube may contain one or more ground, reference, or hypoglossal-monitoring electrode wires wrapped around the outer surface of the endotracheal tube in a circular pattern. Specifically, an uninsulated section of each electrode wire forming a circular pattern is wrapped around the circumference of the endotracheal tube at a different longitudinal location along the endotracheal tube than any other electrode wire forming a circular pattern. From the circular wrapped section, an insulated section of each electrode wire forming a circular pattern runs up the endotracheal tube so that the each electrode wire forming a circular pattern can be connected to an EMG processing machine or nerve stimulator by an electrical connecting plug or to a self-contained indicator that produces at least one of a sound or a light upon receiving certain electrical signals from the electrode wire. Electrode wires dedicated to the same purpose (ground, reference, or hypoglossal-monitoring) may be used to form the circular pattern or a combination of the various purposed electrode wires may be used. The order in which the electrode wires of different purposes are wrapped around the outer surface of the endotracheal tube in a circular pattern may vary.

In other embodiments, the tube may contain ground, reference, or hypoglossal-monitoring electrode wires arranged in an interposing pattern. Electrode wires directed to the same purpose (ground, reference, or hypoglossal-monitoring) may be used to form the interposed pattern or a combination of the various purposed electrode wires may be used. To achieve the interposing pattern, a first primary wire and a second primary wire run parallel to one another and to the central axis of the endotracheal tube from a location on the tube closer to the proximal end of the uninsulated second wire portion than the proximal end of the endotracheal tube, over the insulated first wire portion, and to a location closer to the proximal end of the endotracheal tube than to the proximal end of the uninsulated second wire portion. As used herein, the term “parallel” is intended to describe wires that run in generally the same direction. It may be the case that a projection of the two wires that are considered within the definition of the term “parallel” as used herein would eventually result in the wires intersecting, approaching one another, or diverging from one another, but at least along the length of the endotracheal tube of the present disclosure, the wires described as being “parallel” do not intersect or overlap, and do not approach or diverge from one another to such an extent as to cause cross-talk or interference with one another. A first set of branch wires having the same purpose as the first primary wire run in a first annular direction around a portion of the circumference of the endotracheal tube with each branch wire connected to the first primary wire at spaced intervals. A second set of branch wires having the same purpose as the second primary wire run in a second annular direction around a portion of the circumference of the endotracheal tube with each branch wire connected to the second primary wire at spaced intervals. The first set of branch wires and the second set of branch wires are interposed with one another such that the branch wires do not overlap and are parallel to one another. The length of each branch wire is less than the longer annular distance between the parallel primary electrode wires, such that the branch wires do not cross the primary wires.

When the endotracheal tube is properly positioned in the trachea of a human patient, the uninsulated second wire portion is positioned on the tube so that it contacts a set of laryngeal muscles, particularly a vocal cord of that set of muscles. The uninsulated second wire portion must be long enough so that contact with the laryngeal muscles can be easily accomplished but should not be so long so as to contact other parts of the patient's anatomy. This positioning allows the uninsulated second wire portion to monitor the laryngeal nerves if dedicated to that purpose. However, the electrode wire having the uninsulated second wire portion may function as a ground or reference electrode wire when not being used to monitor the laryngeal nerves. If present, the helical, circular, or interposed pattern of electrode wires ensures contact with the tongue regardless of tube rotation or position in the airway, even if the endotracheal tube rotates after placement, allowing consistently reliable electomyographic signal reading. If present, the helical, circular, or interposed pattern of reference electrode wires can be utilized in conjunction with a nerve stimulator or current emitting probe for nerve-locating and monitoring purposes during surgery. If present, the helical, circular, or interposed pattern of ground and/or reference electrode wires can replace other ground and/or reference electrodes that would otherwise need to be placed elsewhere on the human body during standard electro-physiologic nerve monitoring. Embodiments within the scope of the present invention serve to increase the reliability and decrease the complexity and cost of intra-operative laryngeal as well as hypoglossal nerve monitoring and to lower the morbidity associated with various routine surgical procedures.

In some embodiments within the scope of the present invention, the electrode wires plug into a universal jack. The universal jack can then be connected to an EMG processing machine. One benefit of the universal jack is that it organizes the electrode wires and simplifies the process of plugging the wires into an EMG processing machine. Another benefit is that the EMG processing machine may be programmed such that the purpose (ground, reference, or hypoglossal-monitoring) of each electrode wire may be determined by the EMG machine. Thus, the wires do not have to have their purpose pre-designated and no care need be taken that the correct purposed wire is plugged into the correct electrical connecting plug. The distinct ports and/or electrode wire connections to the universal jack can also be individually labeled to facilitate connections, organization, and troubleshooting.

As opposed to being connected to a separate external EMG machine, one or more electrode wires may be plugged into a self-contained indicator or multiple self-contained indicators that produce a sound, a light, or both upon receiving certain electrical signals. Each self-contained indicator may amplify an electrical signal received from an electrode wire with an amplifier, filter the electrical signal with a filter, process the electrical signal with a processor to determine whether the electrical signal indicates that a nerve is being contacted, and, in the event that the self-contained indicator determines that a nerve is being contacted, indicate nerve contact to a designated observer via a light, sound, or other indicating method. The self-contained indicator may be located on the endotracheal tube, may be located remotely from the endotracheal tube, or may have portions on the endotracheal tube and portions remote from the endotracheal tube. The self-contained indicator may be contained within the same housing as a universal jack. If the self-contained indicator indicates nerve contract by producing only a sound, the entirety of the self-contained indicator may or may not be located on the endotracheal tube. However, if the self-contained indicator indicates nerve contact by producing a light, at least an indicating portion of the self-contained indicator must be far enough removed from the endotracheal tube to be visible to designated observers when the endotracheal tube is properly placed in a patient. The self-contained indicator is advantageous because it provides the necessary feedback for nerve monitoring without a separate, external EMG machine.

### BRIEF DESCRIPTION OF THE DRAWINGS

FIG. 1 is a side view of an endotracheal tube for nerve monitoring having four ground, reference, or laryngeal-monitoring electrode wires running in a direction parallel to the central axis of the tube (two of which are depicted), and two ground electrode wires or, alternately, two hypoglossal-monitoring electrode wires distributed in a helical pattern around the tube.

FIG. 2 is a side view of an endotracheal tube for nerve monitoring having four ground, reference, or laryngeal-monitoring electrode wires running in a direction parallel to the central axis of the tube (two of which are depicted), and two ground electrode wires and two hypoglossal-monitoring electrode wires distributed in a helical pattern around the tube.

FIG. 3 is a side view of an endotracheal tube for nerve monitoring having four ground, reference, or laryngeal-monitoring electrode wires running in a direction parallel to the central axis of the tube (two of which are depicted), and two ground electrode wires and two hypoglossal-monitoring electrode wires distributed in a circular pattern around the tube.

FIG. 4A is a side view of an endotracheal tube for nerve monitoring having four ground, reference, or laryngeal-monitoring electrode wires running in a direction parallel to the central axis of the tube (two of which are depicted), and two primary electrode wires connected to two sets of branch electrode wires distributed in an interposed pattern around the tube.

FIG. 4B is a rear view of the endotracheal tube depicted in FIG. 4A, showing the interposed pattern of the first primary wire and first set of branch wires with the second primary wire and second set of branch wires.

FIG. 5A is a side view of an endotracheal tube for nerve monitoring identical to that depicted in FIG. 1A except that the electrode wires are plugged in to a universal jack unattached to the endotracheal tube.

FIG. 5B is a perspective view of the electrode wires depicted in FIG. 5A plugged into a universal jack.

FIG. 6 is a side view of an endotracheal tube for nerve monitoring identical to that depicted in 5A except that the electrode wires are plugged in to a universal jack attached to the endotracheal tube.

FIG. 7 is a schematic view of actions that may be taken by a self-contained indicator.

### DETAILED DESCRIPTION OF THE DRAWINGS

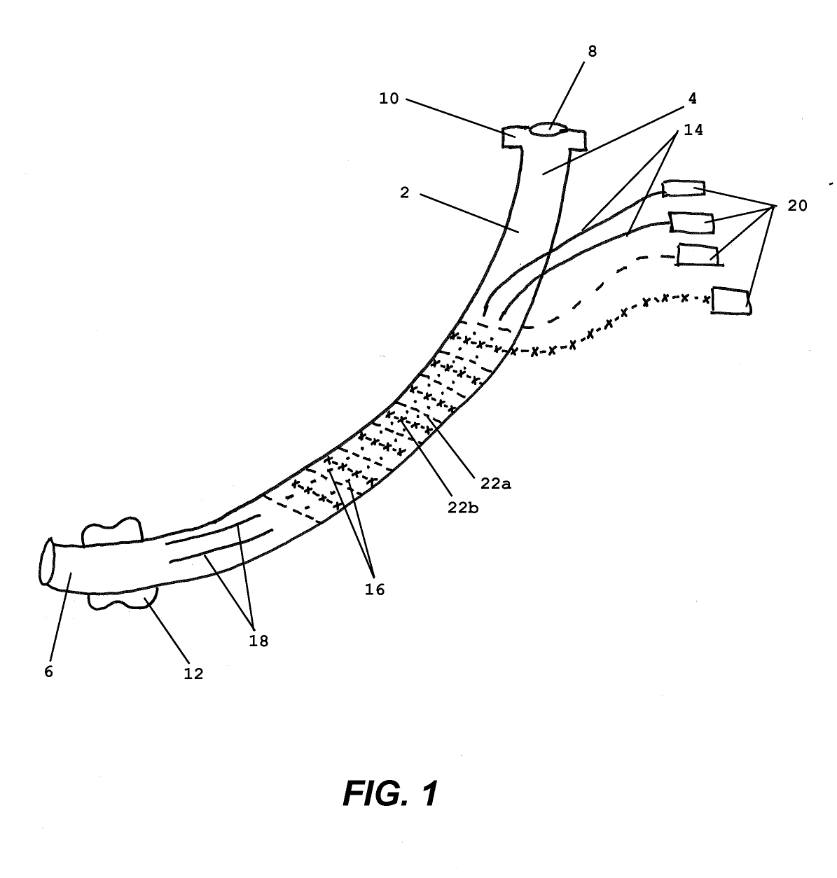

FIGS. 1-4A depict an endotracheal tube 2 made from a flexible, non-electrically conducting material having a proximal end 4 and a distal end 6. The tube 2 has a main lumen 8 for transporting gases to and from the lungs. At proximal end 4 is a fitting 10 for connecting tube 2 to a respiration machine (not depicted) which injects and withdraws air from the lungs. A cuff 12 is located near the distal end 6 and shown in an uninflated condition. The cuff 12 can be inflated by use of a cuff inflating conduit (not depicted) which is attached to a source of compressed air (not depicted) by a fitting (not depicted). In the embodiments depicted in FIGS. 1-4A, four ground, reference, or laryngeal-monitoring electrode wires 14, two of which are depicted, run in a direction parallel to the central axis of the tube 2. The electrode wires 14 are composed of an electrically conductive material, which may include metal paint or other substance printed on the endotracheal tube like ink, metallic tape, or metal strips. Each electrode wire 14 has a first wire portion 16 located between the proximal end 4 and distal end 6 of tube 2 that is insulated against electrical contact.

In the embodiments depicted in FIGS. 1-4A, each first portion 16 is embedded within or coated on the wall of the tube 2 to insulate the first laryngeal-monitoring wire portion 16 from electrical conduct. A second wire portion 18 is located between distal end 6 and first portion 16 on the exterior surface of endotracheal tube 2. Each second wire portion 18 is uninsulated and capable of forming an electrical contact. Electrical connecting plugs 20 are used to connect electrode wires 14 to an EMG processing machine or nerve stimulator (not shown). Any means capable of forming electrical contact such as ports, alligator clips, or insulated wires with bared ends could be used with the present invention instead of the depicted plugs. Alternately, instead of being connected to an EMG machine, each electrode wire 14 may be connected to a self-contained indicator (not depicted in FIGS. 1-4A) that may amplify an electrical signal received from an electrode wire 14 with an amplifier, filter the electrical signal with a filter, process the electrical signal with a processor to determine whether the electrical signal indicates that a nerve is being contacted, and, in the event that the self-contained indicator determines that a nerve is being contacted, indicate nerve contact to a user via a light or sound.

FIG. 1 depicts a tube 2 further comprising two electrode wires 22a (depicted as a purely dash line) and 22b (depicted as an X and dash line) wrapped around the outer surface of the endotracheal tube 2 in a helical pattern. In other embodiments (not shown), more than two or less than two electrode wires 22 may be used to create the helical pattern. The electrode wires 22a and 22b may be ground electrode wires, reference electrode wires through which a nerve stimulator may act, or hypoglossal electrode wires for monitoring of the hypoglossal nerve. The electrode wires 22 include an electrically conductive material, which may include metal paint or other substance printed on the endotracheal tube like ink, metallic tape, or metal strips. In the embodiment depicted in FIG. 1, both electrode wires 22a and 22b are directed to the same purpose (ground, reference, or hypoglossal-monitoring), and specifically in FIG. 1, both electrode wires 22a and 22b are ground electrode wires. In other embodiments, the electrode wires 22a and 22b may be directed to different purposes. Each electrode wire 22 has an identical helical pattern that is offset from the helical pattern of every other electrode wire 22 by a longitudinal distance along tube 2, such that a constant distance is maintained between each of the electrode wires 22 and no crossing of electrode wires 22 occurs. The helical pattern of each wire 22 begins closer to the proximal end of the second wire portions 18 than to the proximal end 4 of the endotracheal tube 2, wraps around the endotracheal tube 2 over the first wire portions 16, and terminates closer to the proximal end 4 of the endotracheal tube 2 than to the proximal end of the second wire portions 18. Electrical connecting plugs 20 may be used to connect the electrode wires 22 to an EMG processing machine or nerve stimulator (not shown). Any means capable of forming electrical contact such as ports, alligator clips, or insulated wires with bared ends could be used with the present invention instead of the depicted plugs. Alternately, instead of being connected to an EMG machine, each electrode wire 22 may be connected to a self-contained indicator (not depicted in FIG. 1) that may amplify an electrical signal received from an electrode wire 22 with an amplifier, filter the electrical signal with a filter, process the electrical signal with a processor to determine whether the electrical signal indicates that a nerve is being contacted, and, in the event that the self-contained indicator determines that a nerve is being contacted, indicate nerve contact to a user via a light or sound.

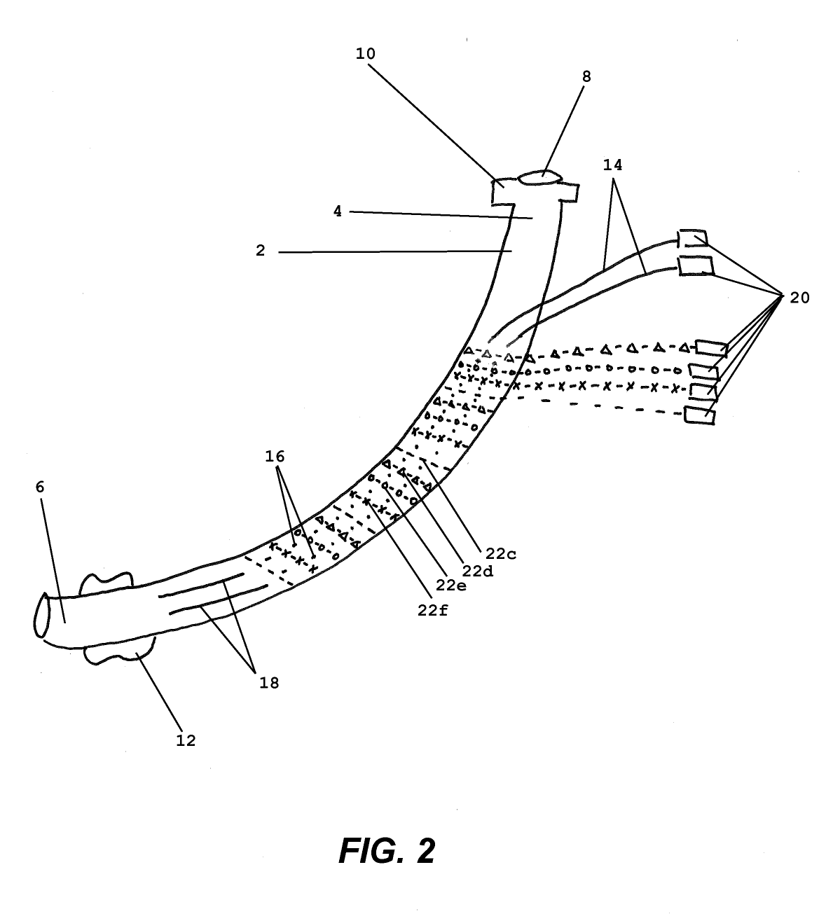

FIG. 2 depicts an embodiment having a helical variation on the helical pattern depicted in FIG. 1. In FIG. 2, four electrode wires 22 (specifically, 22c (depicted as a purely dash line), 22d (depicted as a triangle and dash line), 22e (depicted as a circle and dash line), and 22f (depicted as an X and dash line)) are wrapped around the outer surface of the endotracheal tube 2 in a helical pattern. Two of the electrode wires, 22d and 22f, are ground electrode wires, and the other two electrode wires, 22c and 22e, are hypoglossal-monitoring electrode wires. In this embodiment, the ground electrode wires 22d and 22f are alternated with the hypoglossal-monitoring electrode wires 22c and 22e, such that the closest electrode wire 22 to any other electrode wire 22 serves a different purpose. As discussed with respect to FIG. 1, the number of electrode wires 22 and their purpose (ground, reference, or hypoglossal-monitoring) may be varied. In addition, the order in which the electrode wires 22 of different purposes are wrapped around the outer surface of the endotracheal tube 2 in a helical pattern may vary.

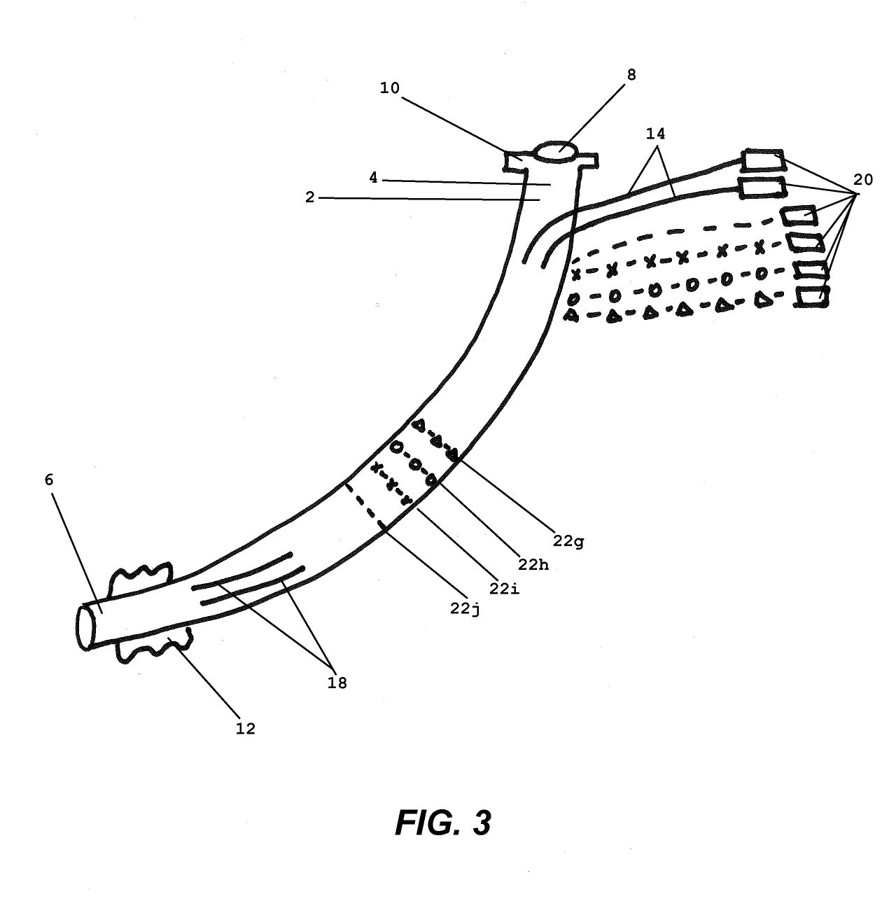

FIG. 3 depicts an embodiment having a circular pattern as opposed to the helical pattern of FIGS. 1 and 2. In FIG. 3, four electrode wires 22 (specifically, 22g (depicted as a triangle and dash line), 22h (depicted as a circle and dash line), 22i (depicted as an X and dash line), and 22j (depicted as a purely dash line)) are each wrapped around the outer surface of the endotracheal tube 2 in a circular pattern. Specifically, an uninsulated section of each electrode wire 22 is wrapped around the circumference of the endotracheal tube 2 at a different longitudinal location along the endotracheal tube 2 than any other electrode wire 22. From the circular wrapped section, an insulated section of each electrode wire 22 runs up the endotracheal tube 2 so that the each electrode wire 22 can be connected to an EMG processing machine or nerve stimulator by an electrical connecting plug 20. Insulation of the insulated section of each electrode wire 22 can be achieved by coating the electrode wire 22 or embedding it within the endotracheal tube 2._Any means capable of forming electrical contact such as ports, alligator clips, or insulated wires with bared ends could be used with the present invention instead of the depicted plugs. Alternately, instead of being connected to an EMG machine, each electrode wire 22 may be connected to a self-contained indicator (not depicted in FIG. 3) that may amplify an electrical signal received from an electrode wire 22 with an amplifier, filter the electrical signal with a filter, process the electrical signal with a processor to determine whether the electrical signal indicates that a nerve is being contacted, and, in the event that the self-contained indicator determines that a nerve is being contacted, indicate nerve contact to a user via a light or sound. In the embodiment depicted in FIG. 3, two electrode wires (22g and 22i) are ground electrode wires and two electrode wires (22h and 22j) are hypoglossal-monitoring wires. In other embodiments, different numbers of wires may be used, and the wires may all be directed to the same purpose or may be directed to more than one purpose. In the embodiment depicted in FIG. 3, the ground electrode wires 22g and 22i are alternated with the hypoglossal-monitoring electrode wires 22h and 22j, such that the closest electrode wire 22 to any other electrode wire 22 serves a different purpose. In other embodiments, the order in which the electrode wires 22 of different purposes are wrapped around the outer surface of the endotracheal tube 2 in a circular pattern may vary.

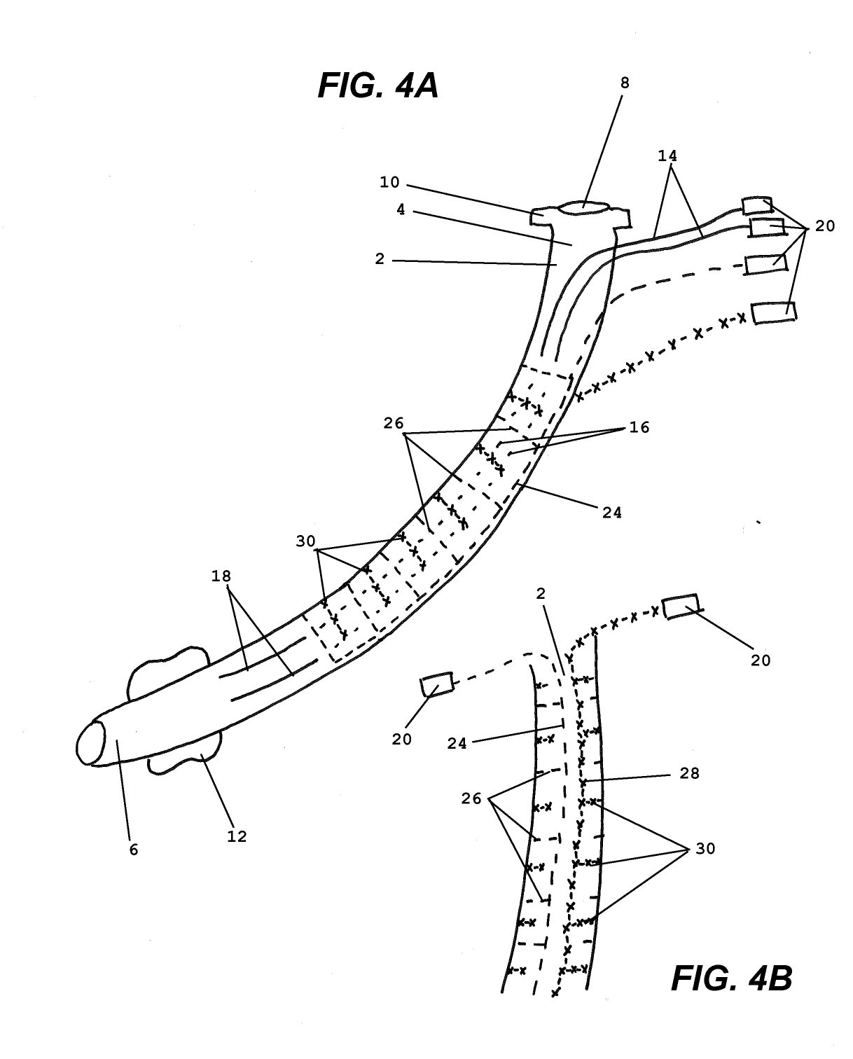

FIGS. 4A and 4B depict a tube 2 with an interposing pattern formed by first primary electrode wire 24 (depicted as a purely dash line), a first set of branch electrode wires 26 (also depicted as a purely dash line), second primary electrode wire 28 (depicted as an X and dash line), and second set of branch electrode wires 30 (also depicted as an X and dash line), all of which are composed of an electrically conductive material, which may include metal paint or other substance printed on the endotracheal tube like ink, metallic tape, or metal strips. As shown best in FIG. 4B, the first primary wire 24 and second primary wire 28 run parallel to one another and to the central axis of the endotracheal tube 2 on the outer surface of the endotracheal tube 2 from a location on the endotracheal tube 2 closer to the proximal end of the uninsulated second wire portions 18 than to the proximal end 4 of the endotracheal tube 2, over the insulated first wire portions 16, and to a location closer to the proximal end 4 of the endotracheal tube 2 than to the proximal end of the uninsulated second wire portions 18. A first set of branch wires 26, of the same purpose (ground, hypoglossal-monitoring, or reference) as the first primary electrode wire 24, run in a first annular direction around a portion of the circumference of the endotracheal tube 2 on the outer surface of the endotracheal tube 2 with each branch wire of the first set of branch wires 26 connected to the first primary wire 24 at spaced intervals. A second set of branch wires 30, of the same purpose (ground, hypoglossal-monitoring, or reference) as the second primary electrode wire 28, run in a second annular direction that is opposite the first annular direction around a portion of the circumference of the endotracheal tube 2 on the outer surface of the endotracheal tube 2 with each of the branch wires of the second set of branch wires 30 connected to the second primary wire 28 at spaced intervals. The first set of branch wires 26 and the second set of branch wires 30 are interposed with one another such that they do not overlap and are parallel to one another. The length of each branch wire of the first set 26 and second set 30 is less than the longer annular distance between the parallel primary electrode wires 24 and 28, such that none of the branch wires of the first set 26 cross the second primary electrode wire 28 and none of the branch wires of the second set 30 cross the first primary electrode wire 24. With respect to the embodiment disclosed in FIGS. 4A and 4B, the first primary electrode wire 24 and first set of branch wires 26 may have the same or a different purpose than the second primary electrode wire 28 and second set of branch wires 30. For example, the first primary electrode wire 24 and first set of branch wires 26 may be ground wires and the second primary electrode wire 28 and second set of branch wires 30 may be hypoglossal-monitoring wires. Alternately, the first primary electrode wire 24, first set of branch wires 26, second primary electrode wire 28, and second set of branch wires 30 may all be ground wires. Electrical connecting plugs 20 may be used to connect the first primary electrode wire 24 and second primary electrode wire 28 to an EMG processing machine or nerve stimulator (not shown). Any means capable of forming electrical contact such as ports, alligator clips, or insulated wires with bared ends could be used with the present invention instead of the depicted plugs. Alternately, instead of being connected to an EMG machine, first primary electrode wire 24 and second primary electrode wire 28 may be connected to a self-contained indicator (not depicted in FIGS. 4A and 4B) that may amplify an electrical signal received from first primary electrode wire 24 or second primary electrode wire 28 with an amplifier, filter the electrical signal with a filter, process the electrical signal with a processor to determine whether the electrical signal indicates that a nerve is being contacted, and, in the event that the self-contained indicator determines that a nerve is being contacted, indicate nerve contact to a user via a light or sound.

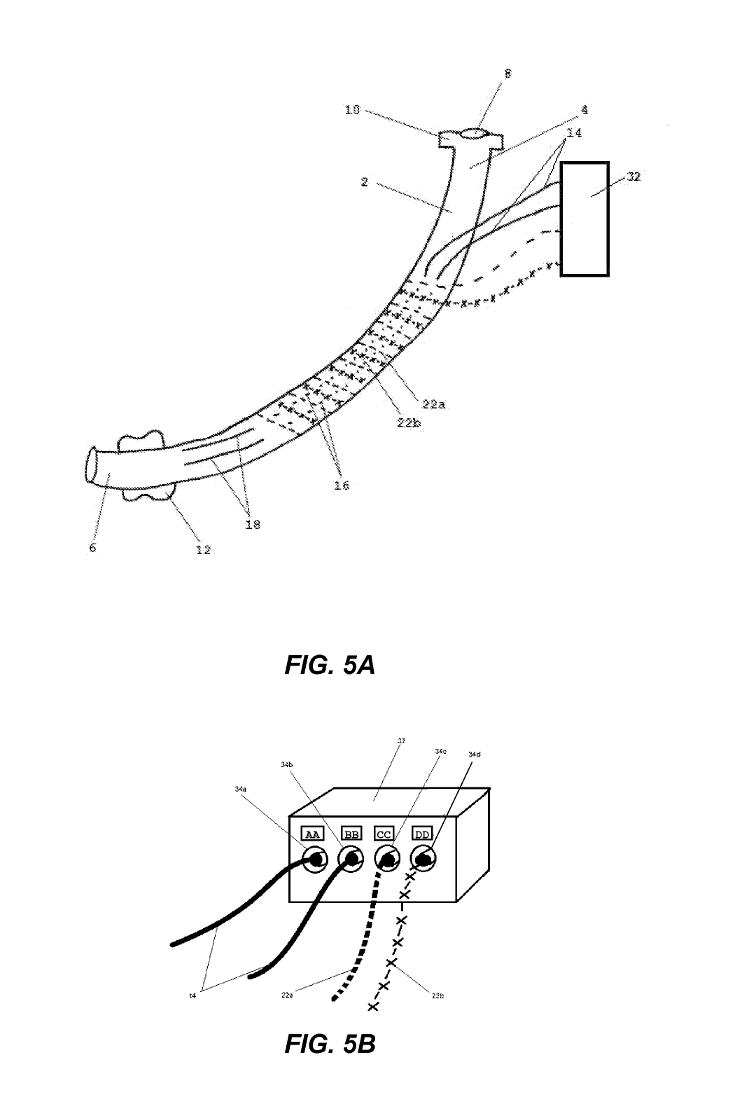

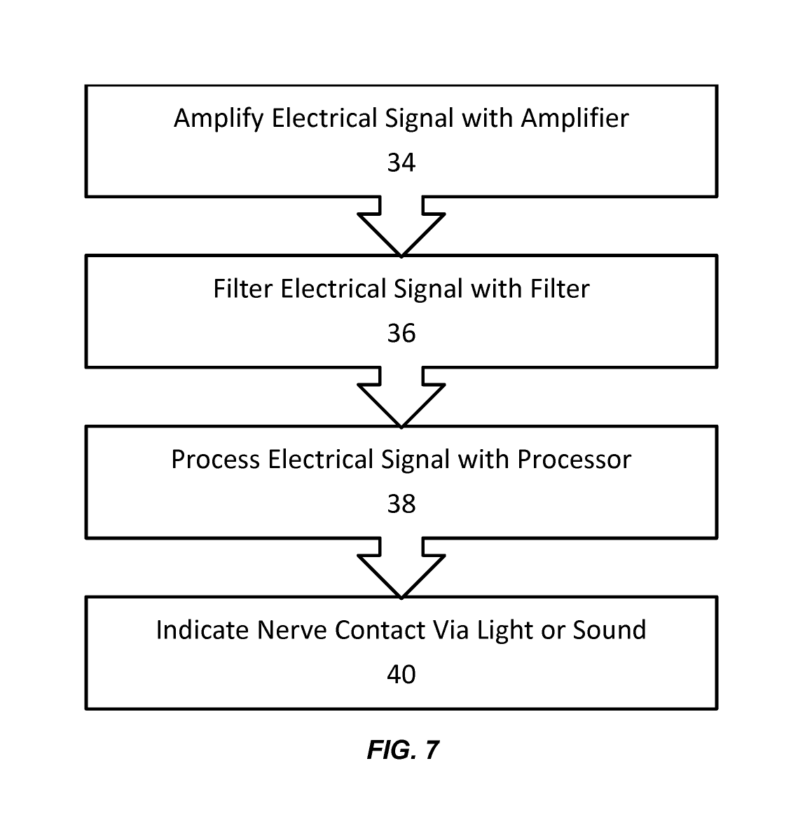

FIG. 5A shows the endotracheal tube 2 depicted in FIG. 1 with the electrode wires 14, 22a, and 22b connected to a universal jack 32 rather than electrical connecting plugs 20. The universal jack 32 depicted in FIG. 5A is not attached directly to the endotracheal tube 2 directly. Although the embodiment depicted in FIG. 1 has four electrode wires (of which, two electrode wires 14 are depicted) running in a direction parallel to the central axis of the tube 2 and two electrode wires 22a and 22b distributed in a helical pattern around the tube 2, a universal jack 32 could easily be used with other patterns or arrangements of electrode wires, such as an interposed pattern of electrode wires. The universal jack 32 may be connected to an EMG processing machine. The EMG processing machine may assign the electrode wires 14, 22a, and 22b a purpose (ground, reference, or hypoglossal-monitoring) via the universal jack 32. The universal jack 32 may help to organize the electrode wires 14, 22a, and 22b and may make it easier to plug the electrode wires into an EMG processing machine. To further facilitate such organization, the individual ports of the universal jack and/or electrode wires can be labeled. Alternately, the universal jack 32 may include a self-contained indicator, that may be used in accordance with the method disclosed in FIG. 7. Because the universal jack 32 in FIG. 5A is not directly attached to the endotracheal tube 2, a self-contained indicator included with the universal jack 32 could indicate nerve contact via light or sound.

FIG. 5B shows the electrode wires 14, 22a, and 22b plugged into the universal jack 32 at jack connections 34a, 34b, 34c, and 34d. The universal jack 32 may have additional jack connections 34 to accommodate other electrode wires, such as the electrode wires in FIG. 5A running in a direction parallel to the central axis of the tube 2 that are not depicted. The jack connections 34 may be clustered together on one portion of the universal jack 32 or may be spread out over the universal jack 32. The universal jack 32 further comprises an EMG processing machine connection, not depicted.

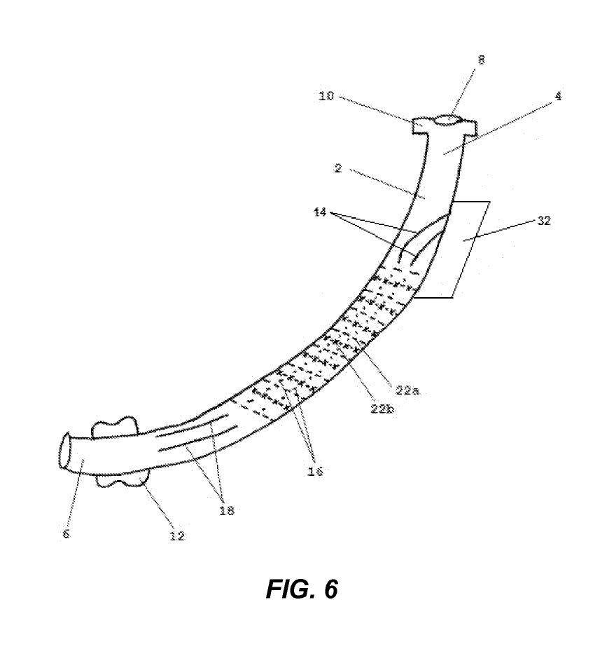

FIG. 6 shows the endotracheal tube 2 depicted in FIG. 5A with the universal jack 32 attached directly to the endotracheal tube 2. Because the universal jack 32 in FIG. 6 is directly attached to the endotracheal tube 2, a self-contained indicator included with the universal jack 32 could indicate nerve contact only via sound or another indicating method where visibility is not required because a light would not be visible when the endotracheal tube 2 was properly positioned in a patient. In other embodiments within the scope of the present disclosure not herein depicted, a self-contained indicator contained with the universal jack 32 that is attached directly to an endotracheal tube 2 could have an unattached indicating portion that would be visible when the endotracheal tube 2 was properly positioned in a patient so that light could be used to indicate nerve contact. The universal jack 32, which may include a self-contained indicator, may have a variety of shapes in embodiments within the scope of the present disclosure. For example, in an embodiment, the universal jack 32 including a self-contained indicator may be cylindrical and surround the entire circumference of endotracheal tube 2. In another embodiment within the scope of the present disclosure, the universal jack 32 including a self-contained indicator may only be located on one side of the endotracheal tube 2. In some embodiments within the scope of the present disclosure, the universal jack 32 including the self-contained indicator may be separate but attachable to the endotracheal tube 2, such that the endotracheal tube may be inserted into a patient and then the separate universal jack 32 including the self-contained indicator may be attached to the endotracheal tube 2.

FIG. 7 illustrates a schematic view of a method of operation of a self-contained indicator. First, as shown at 34, the self-contained indicator may amplify an electrical signal received from an electrode wire 14, 22, 24 or 28 or from universal jack 32. The self-contained indicator may then filter the electric signal as shown at 36. The electric signal may then be processed by a processor of the self-contained indicator as shown at 38 to determine whether a nerve has been contacted. Finally, as shown at 40, if the self-contained indicator determines that a nerve has been contacted, the self-contained indicator may indicate nerve contract via a light or sound or other indicating method. The self-contained indicator may comprise an indicating portion for purposes of generating the light, sound, or other indicating method, and the indicating portion may or may not be integral with the rest of the self-contained indicator.

While the present disclosure has been described with respect to certain embodiments, it will be understood that variations may be made thereto that are still within the scope of the appended claims.

What is claimed is:

1. A nerve monitoring device comprising:
an endotracheal tube formed from a flexible, non-electrically conducting material and having a distal end, a proximal end, a central axis, and an outer surface;
at least one ground, reference, or laryngeal-monitoring electrode wire including electrically conducting material running in a direction parallel to the central axis at a location between the distal end and the proximal end of the endotracheal tube;
the at least one ground, reference, or laryngeal-monitoring electrode wire having an electrically insulated first wire portion located between the distal end and proximal end of the endotracheal tube and an electrically uninsulated second wire portion located on the outer surface of the endotracheal tube between the first wire portion and the distal end of the endotracheal tube;
the second wire portion comprising means for contacting the laryngeal muscles when the endotracheal tube is placed in the trachea for ventilation; and
at least one ground, reference, or hypoglossal-monitoring electrode wire including electrically conducting material wrapped around the outer surface of the endotracheal tube in a helical or circular pattern.

2. The device of claim 1, wherein the pattern of each of the at least one ground, reference, or hypoglossal-monitoring electrode wire wrapped around the outer surface of the endotracheal tube is helical and begins close to the second wire portion than to the proximal end of the endotracheal tube, wraps over the first wire portion, and terminates closer to the proximal end of the endotracheal tube than to the second wire portion.

3. The device of claim 1, wherein at least two ground, reference, or hypoglossal-monitoring electrode wires are wrapped around the outer surface of the endotracheal tube in a helical or circular pattern.

4. The device of claim 3, wherein the pattern of each of the at least two ground, reference, or hypoglossal-monitoring electrode wires wrapped around the outer surface of the endotracheal tube is helical and is identical and each of the at least two ground, reference, or hypoglossal-monitoring electrode wires having a helical pattern is offset from every other wire having a helical pattern by a longitudinal distance along the endotracheal tube.

5. The device of claim 2, wherein all of the ground, reference, or hypoglossal-monitoring electrode wires wrapped around the outer surface of the endotracheal tube are dedicated to the same purpose.

6. The device of claim 2, wherein not all of the ground, reference, or hypoglossal-monitoring electrode wires wrapped around the outer surface of the endotracheal tube are dedicated to the same purpose.

7. The device of claim 6, wherein the order in which the ground, reference, or hypoglossal-monitoring electrode wires wrapped around the outer surface of the endotracheal tube are wrapped around the outer surface of the endotracheal tube is alternated such that at least one ground, reference, or hypoglossal-monitoring electrode is next to another ground, reference, or hypoglossal-monitoring electrode of a different purpose.

8. The device of claim 1, wherein the first wire portion is coated or embedded within the wall of the endotracheal tube.

9. The device of claim 5, wherein the at least two ground, reference, or hypoglossal-monitoring electrode wires wrapped around the outer surface of the endotracheal tube consist of two ground electrode wires.

10. The device of claim 7, wherein the at least two ground, reference, or hypoglossal-monitoring electrode wires wrapped around the outer surface of the endotracheal tube consist of two ground electrode wires and two hypoglossal-monitoring electrode wires and the ground electrode wires are alternated with the hypoglossal-monitoring wires.

11. The device of claim 1, wherein the at least one ground, reference, or laryngeal-monitoring electrode wire including electrically conducting material running in a direction parallel to the central axis is either a ground or reference electrode wire.

12. The device of claim 1, further comprising an electrical connecting means for attaching at least one of the ground, reference, or laryngeal-monitoring electrode wires to a machine which processes EMG signals.

13. The device of claim 1, further comprising a self-contained indicator connected to at least one of the ground, reference, laryngeal-monitoring, or hypoglossal-monitoring electrode wires, wherein the self-contained indicator produces at least one of a sound or a light upon receiving certain electrical signals.

14. The device of claim 13, wherein the self-contained indicator includes at least one of an amplifier, a filter, and a processor.

15. A nerve monitoring device comprising:
an endotracheal tube formed from a flexible, non-electrically conducting material and having a distal end, a proximal end, a central axis, and an outer surface;
at least one ground, reference, or laryngeal-monitoring electrode wire including electrically conducting material running in a direction parallel to the central axis at a location between the distal end and the proximal end of the endotracheal tube;
the at least one electrode wire having an electrically insulated first wire portion located between the distal end and proximal end of the endotracheal tube and an electrically uninsulated second wire portion located on the outer surface of the endotracheal tube between the first wire portion and the distal end of the endotracheal tube; and
the second laryngeal monitoring wire portion comprising means for contacting the laryngeal muscles when the endotracheal tube is placed in the trachea for ventilation;
a first primary electrode wire including electrically conducting material connected to a first set of branch electrode wires including electrically conducting material and a second primary electrode wire including electrically conducting material connected to a second set of branch electrode wires including electrically conducting material, wherein the first set of branch electrode wires and the second set of branch electrode wires are interposed with one another.

16. The device of claim 15, wherein the first primary electrode wire and the second primary electrode wire run parallel to one another and to the central axis of the endotracheal tube from a location on the tube closer to the second wire portion than to the proximal end of the endotracheal tube, over the first wire portion, and to a location closer to the proximal end of the endotracheal tube than to the second wire portion.

17. The device of claim 16, wherein the first primary electrode wire and the first set of branch electrode wires have the same purpose of ground, reference, or hypoglossal-monitoring as each other, and the second primary electrode wire and second set of branch electrode wires have the same purpose of ground, reference, or hypoglossal-monitoring as each other.

18. The device of claim 17, wherein the first set of branch electrode wires run in a first annular direction around a portion of the circumference of the endotracheal tube and the second set of branch electrode wires run in a second annular direction that is opposite the first annular direction around a portion of the circumference of the endotracheal tube.

19. The device of claim 18, wherein the length of each branch electrode wire of the first set of branch electrode wires and the second set of branch electrode wires is less than the longer annular distance between the first primary electrode wire and the second primary electrode wire.

20. The device of claim 19, wherein each branch electrode wire of the first set of branch electrode wires and the second set of branch electrode wires is parallel to every other branch electrode wire of the first set of branch electrode wires and the second set of branch electrode wires.

21. The device of claim 17, wherein the first primary electrode wire and first set of branch electrode wires have the same purpose as the second primary electrode wire and second set of branch electrode wires.

22. The device of claim 17, wherein the first primary electrode wire and first set of branch electrode wires have a different purpose than the second primary electrode wire and second set of branch electrode wires.

23. The device of claim 19, wherein the first primary electrode wire and first set of branch electrode wires are ground wires and the second primary electrode wire and second set of branch electrode wires are hypoglossal-monitoring wires.

24. The device of claim 15, wherein the first wire portion is coated or embedded within the wall of the endotracheal tube.

25. The device of claim 15, wherein the at least one ground, reference, or laryngeal-monitoring electrode wire including electrically conducting material running in a direction parallel to the central axis is either a ground or reference electrode wire.

26. The device of claim 15, further comprising an electrical connecting means for attaching at least one of the ground, reference, or laryngeal-monitoring electrode wires to a machine which processes EMG signals.

27. The device of claim 1, wherein the electrical connecting means is a universal jack.

28. The device of claim 26, wherein the electrical connecting means is a universal jack.

31. The device of claim 15, further comprising a self-contained indicator connected to at least one of the ground, reference, laryngeal-monitoring, first primary, or second primary electrode wires, wherein the self-contained indicator produces at least one of a sound or a light upon receiving certain electrical signals.

32. The device of claim 31, wherein the self-contained indicator includes at least one of an amplifier, a filter, and a processor.
